(19) United States
(12) Patent Application Publication
(10) Pub. No.: US 2026/0334900 A1
(43) Pub. Date: Oct. 29, 2026

(54) MEDICAL IMAGING APPARATUS FOR INCLUDING DISPLAY ARM

(71) Applicant: DRTECH CORPORATION, Seongnam-si Gyeonggi-do (KR)

(72) Inventors: Cheon Kyung SUNG, Uijeongbu-si Gyeonggi-do (KR); Choul Woo SHIN, Seongnam-si Gyeonggi-do (KR)

(73) Assignee: DRTECH CORPORATION, Seongnam-si Gyeonggi-do (KR)

(21) Appl. No.: 19/709,690

(22) Filed: Jun. 16, 2026

Related U.S. Application Data
(63) Continuation of application No. PCT/KR2025/020573, filed on Dec. 3, 2025.

(30) Foreign Application Priority Data
Feb. 13, 2025 (KR) 10-2025-0018917

(51) Int. Cl. A61B 6/46; A61B 6/00

(52) U.S. Cl. CPC A61B 6/462; A61B 6/4441; A61B 6/4476

(57) ABSTRACT

The medical imaging apparatus of the present disclosure includes a body, an arm driving unit supporting and rotating a C-arc and coupled to one side of the body, a first display coupled to the arm driving unit, and a second display coupled to the arm driving unit, wherein the first display and the second display rotate independently of each other about a single arm rotation axis.

### CROSS-REFERENCE TO RELATED APPLICATIONS

This application is a continuation of International Application No. PCT/KR2025/020573 filed on Dec. 3, 2025, which claims priority to Korean Patent Application No. 10-2025-0018917 filed on Feb. 13, 2025, the entire contents of which are herein incorporated by reference.

### TECHNICAL FIELD

The present disclosure relates to a medical imaging apparatus including display arms. More specifically, the medical imaging apparatus includes a plurality of display arms, and the plurality of display arms are capable of moving independently.

### BACKGROUND ART

Recently, in the field of medical imaging, the need for precise, high-resolution, real-time images for accurate diagnosis and treatment has been greatly increasing. A C-arm is an imaging apparatus that irradiates radiation onto an affected area of a human or animal body and receives the transmitted radiation to acquire an image of the affected area. Such a radiographic imaging apparatus is a device that continuously or successively provides radiation images of an affected area, and is widely used for diagnosis and interpretation of affected areas and various medical procedures.

Recently, C-ARMs providing real-time images for improved diagnostic accuracy have seen an increase in interventional procedures using fluoroscopy and three-dimensional images during observation and surgery of fractures in orthopedics including the spine, and diagnosis and procedures for disorders of cerebrovascular and nervous systems in neurosurgery, and accordingly, treatment efficiency has been greatly increasing.

In addition, research and development of C-ARMs is being conducted in the direction of improving image quality and significantly reducing radiation exposure, and in particular, research and development is being conducted so that C-ARMs are configured to acquire radiation images from various angles while keeping a patient or subject fixed by adjusting a rotation angle. This is because a C-arm is mobile equipment used mainly in limited, compact spaces such as operating rooms, and thus must be capable of efficiently using space. In addition, during medical imaging, in order to allow medical staff (e.g., doctors, radiologists, etc.) to easily and conveniently manipulate medical imaging information, device information, and data in a simple manner, a C-ARM must allow a plurality of displays to move freely.

### SUMMARY

### Technical Problem

The present disclosure provides a medical imaging apparatus in which an existing complex structure is further simplified, thereby reducing cost, improving assemblability, achieving weight reduction, and facilitating repair.

The present disclosure provides a medical imaging apparatus including display arms capable of positioning each of a plurality of displays at a position desired by a user.

The present disclosure provides a medical imaging apparatus including display arms capable of fixing each of a plurality of displays at a position desired by a user.

### Technical Solution

A medical imaging apparatus according to the present disclosure includes a body, an arm driving unit supporting and rotating a C-arc and coupled to one side of the body, a first display coupled to the arm driving unit, and a second display coupled to the arm driving unit, wherein the first display and the second display rotate independently of each other about a single arm rotation axis.

The medical imaging apparatus according to the present disclosure further includes a first display arm 160 for coupling the first display to the arm driving unit and a second display arm 170 for coupling the second display to the arm driving unit, and the first display arm and the second display arm rotate about the single arm rotation axis so that the first display and the second display each rotate independently.

The second display arm 170 of the medical imaging apparatus according to the present disclosure includes at least one sub-arm.

The medical imaging apparatus according to the present disclosure includes a display arm rotation pole 210 coupled to an arm housing 153 included in the arm driving unit so as to be rotatable with respect to the arm housing, at least a portion of the second display arm is coupled to the display arm rotation pole, and at least a portion of the first display arm is rotatably coupled to the second display arm.

The medical imaging apparatus according to the present disclosure further includes a support block to which at least one of the first display arm and the second display arm is rotatably coupled, and a support block fixing unit 340 for fixing the support block so that the support block is not rotatable.

The medical imaging apparatus according to the present disclosure further includes a support block receiving part 240 coupled to the second display arm 170 and rotatably coupled to the support block, a second wear reduction member 331 located between the support block and an arm shaft 250 coupled to the first display arm 160, a third wear reduction member 510 located between the support block receiving part and the support block, a fourth wear reduction member 350 located between the arm housing 153 and the display arm rotation pole 210, and a first wear reduction member 332 located between at least a portion of a lower surface of the first display arm 160 and at least a portion of an upper surface of the support block 220.

The support block of the medical imaging apparatus according to the present disclosure includes a first support block 221 for supporting at least a portion of a lower portion of the first display arm, a second support block 222 coupled to a lower portion of the first support block, and a third support block 223 located at a lower portion of the second support block and to which the second display arm is rotatably coupled.

The medical imaging apparatus according to the present disclosure further includes a radiation irradiator outputting radiation and irradiating the radiation onto a subject, a radiation detector receiving the radiation transmitted through the subject and acquiring an image signal, a C-arc coupled to the radiation irradiator and coupled to the radiation detector at a position opposite to the radiation irradiator, a stay arm included in the arm driving unit, coupled to the C-arc, and rotating the C-arc orbitally or pivotally, a front-rear driving arm included in the arm driving unit, having the stay arm coupled to one side thereof, and moving the C-arc in at least one of a forward direction, a rearward direction, an upward direction, and a downward direction, an arm housing included in the arm driving unit and located above the front-rear driving arm, and a movement fixing unit for limiting at least one of rotation of the C-arc, rotation of the first display arm, and rotation of the second display arm.

The movement fixing unit of the medical imaging apparatus according to the present disclosure includes a brake lining contacting a rotation target and preventing rotation of the rotation target, and a brake shoe coupled to at least a portion of the brake lining and moving the brake lining toward the rotation target or away from the rotation target.

The brake lining of the medical imaging apparatus according to the present disclosure is a woven liner woven from fiber material.

In addition, a program for implementing the operation method of the medical imaging apparatus as described above may be recorded on a computer-readable recording medium.

### Advantageous Effects

The medical imaging apparatus of the present disclosure further simplifies an existing complex structure, thereby reducing cost, improving assemblability, achieving weight reduction, and facilitating repair.

A user of the medical imaging apparatus of the present disclosure can easily check images captured in real time from any position or angle during a procedure through dual displays and dual arms. Therefore, the medical imaging apparatus of the present disclosure can maximize usability and procedure space efficiency. In addition, the medical imaging apparatus of the present disclosure provides a clamp lever at a joint of a display arm so that a user can quickly and easily fix dual displays during a procedure or parking. Therefore, user convenience can be maximized.

The effects obtainable from the present disclosure are not limited to the effects mentioned above, and other effects not mentioned will be clearly understood by those skilled in the art to which the present disclosure belongs from the description below.

### DESCRIPTION OF DRAWINGS

FIG. 1 is a view illustrating a mobile medical imaging apparatus according to one embodiment of the present disclosure.

FIG. 2 illustrates a cross section of a part of the medical imaging apparatus according to one embodiment of the present disclosure.

FIG. 3 illustrates a cross section of a part of the medical imaging apparatus according to one embodiment of the present disclosure.

FIG. 4 illustrates a cross section of a part of the medical imaging apparatus according to one embodiment of the present disclosure.

FIG. 5 illustrates a cross section of a part of the medical imaging apparatus according to one embodiment of the present disclosure.

FIG. 6 is a view for describing the second display arm according to one embodiment of the present disclosure.

FIG. 7 is a view for describing the display arm according to one embodiment of the present disclosure.

FIG. 8 is a view for describing the display arm according to one embodiment of the present disclosure.

FIG. 9 is a view for describing the display arm according to one embodiment of the present disclosure.

FIG. 10 is a view for describing the movement fixing unit according to one embodiment of the present disclosure.

FIG. 11 is a view for describing the movement fixing unit according to one embodiment of the present disclosure.

FIG. 12 is a view for describing the movement fixing unit according to one embodiment of the present disclosure.

### DETAILED DESCRIPTION

The advantages and features of the disclosed embodiments and methods for achieving the same will become apparent from the following description of the embodiments, taken in conjunction with the accompanying drawings. However, the present disclosure is not limited to the embodiments disclosed below herein but will be implemented in various forms, and the embodiments are provided so that the present disclosure is completely disclosed, and a person of ordinary skill in the art can fully understand the scope of the present disclosure.

Terms used herein will be described in brief, and then the disclosed embodiments will be described in detail.

The terms used herein have been selected as general terms which are widely used at present in consideration of the functions of the present disclosure, and this may be altered according to the intent of an operator skilled in the art, conventional practice, or introduction of new technology. Also, in a specific case, a term is arbitrarily selected by the applicant, and the meaning of the term will be described in detail in a corresponding description portion of the present disclosure. Therefore, the terms used herein should be defined based on the overall content of the present disclosure instead of a simple name of each of the terms.

As used herein, the singular forms “a” and “an” are intended to include the plural forms as well, unless the context clearly indicates the singular form. Further, the plural forms are intended to include the singular forms as well, unless the context clearly indicates the plural forms.

Throughout the specification, unless explicitly described to the contrary, the word “include” and variations such as “comprise” or “comprising” will be understood to imply the inclusion of stated elements but not the exclusion of any other elements.

Further, the term “unit” used herein refers to a software or hardware component, and the “unit” performs certain roles. However, the “unit” is not limited to software or hardware. The “unit” may be formed to be in an addressable storage medium or may be formed to operate one or more processors. Thus, as an example, the term “unit” includes components such as software components, object-oriented software components, class components, and task components, processes, functions, attributes, procedures, subroutines, segments of program code, drivers, firmware, micro-codes, circuits, data, a database, data structures, tables, arrays, and variables. Functions provided in the components and the “units” may be combined into a smaller number of components and “units,” or further divided into additional components and “units.”

According to one embodiment of the present disclosure, the “unit” may be implemented as a processor and memory. The term “processor” should be interpreted broadly to include a general-purpose processor, a central processing unit (CPU), a microprocessor, a digital signal processor (DSP), a controller, a microcontroller, a state machine, or the like. In some environments, the “processor” may also refer to an application-specific integrated circuit (ASIC), a programmable logic device (PLD), a field-programmable gate array (FPGA), or the like. The term “processor” may also refer, for example, to a combination of processing devices such as a combination of a DSP and a microprocessor, a combination of a plurality of microprocessors, a combination of one or more microprocessors in conjunction with a DSP core, or any other combination of such components.

The term “memory” should be interpreted broadly to include any electronic component capable of storing electronic information. The term “memory” may also refer to various types of processor-readable media such as a random access memory (RAM), a read-only memory (ROM), a non-volatile random access memory (NVRAM), a programmable read-only memory (PROM), an erasable-programmable read-only memory (EPROM), an electrically erasable PROM (EEPROM), a flash memory, a magnetic or optical data storage, and registers. The memory is said to be in electronic communication with the processor when the processor can read information from the memory and/or write information to the memory. The memory integrated into the processor is in electronic communication with the processor.

Hereinafter, embodiments will be described in detail with reference to the accompanying drawings so that those skilled in the art may easily practice the present disclosure. In order to clearly describe the present disclosure with reference to the drawings, parts that are not related to the description are omitted.

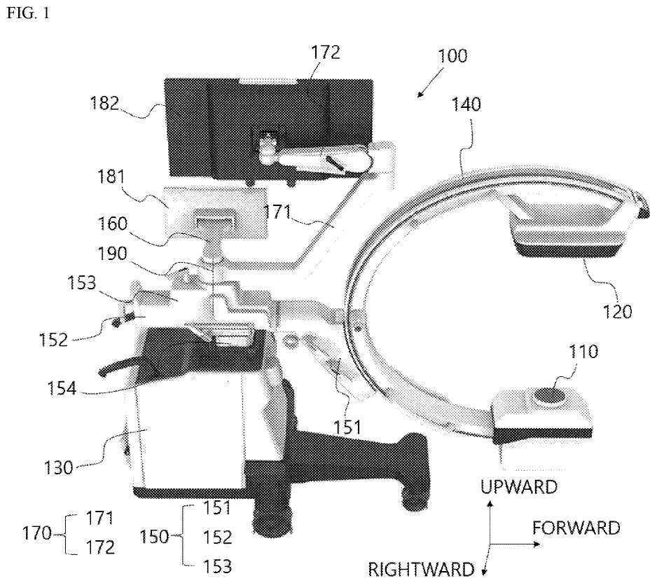

FIG. 1 is a view illustrating a mobile medical imaging apparatus according to one embodiment of the present disclosure.

Referring to FIG. 1, the mobile medical imaging apparatus 100 of the present disclosure may include wheels and thus may be movable. However, the present disclosure is not limited thereto, and the medical imaging apparatus 100 may be a fixed apparatus.

The medical imaging apparatus 100 according to one embodiment may be an apparatus capable of imaging and/or examining an internal structure of a subject (or object) based on radiation including X-rays. For example, the medical imaging apparatus irradiates X-rays to transmit through the human body and scans the transmitted X-rays to acquire an image of the inside of the human body.

Referring to FIG. 1, the medical imaging apparatus 100 may include a radiation irradiator 110, a radiation detector 120, and a body 130. In addition, the body 130 of the medical imaging apparatus 100 may include a high-voltage generator (not shown in the drawings) and/or a controller. The body 130 may be coupled to at least one of an output unit and an input unit.

The body 130 may be movable. The body 130 may include wheels. The wheels may include at least one of a caster wheel, a motorized wheel, and an omni wheel. The body 130 may be moved by a user's force or may be automatically moved by a wheel actuator.

The radiation irradiator 110 may be a component outputting radiation and irradiating the radiation onto a subject. The radiation detector 120 may be a component receiving the radiation transmitted through the subject and acquiring an image signal.

A user may move the medical imaging apparatus 100 close to a patient bed. The user may place the radiation detector 120 behind the subject. Therefore, the radiation irradiated from the radiation irradiator 110 may pass through the subject and reach the radiation detector 120. The radiation detector 120 may detect the radiation that has passed through the subject and convert the radiation into an electrical signal. In addition, the radiation detector 120 may acquire a radiation image based on the electrical signal.

The medical imaging apparatus 100 may include a C-arc 140. The C-arc 140 may be coupled to the radiation irradiator 110. In addition, the C-arc 140 may be coupled to the radiation detector 120 at a position opposite to the radiation irradiator 110. Referring to FIG. 1, the radiation irradiator 110 may be coupled to a lower side of the C-arc 140, and the radiation detector 120 may be coupled to an upper side of the C-arc 140.

The C-arc 140 may move in at least one of forward, rearward, upward, and downward directions by at least one of the body 130 and the arm driving unit 150, and rotational rotation and orbital rotation may also be possible. Since the C-arc 140 is capable of moving with a plurality of degrees of freedom, the C-arc 140 may be positioned at an optimal position with respect to the patient to image the affected area of the patient. Therefore, the medical imaging apparatus 100 of the present disclosure helps a user make an accurate diagnosis.

The medical imaging apparatus 100 may include the arm driving unit 150. The arm driving unit 150 may support the C-arc 140. In addition, the arm driving unit 150 may rotate the C-arc 140. The arm driving unit 150 may include at least one of a stay arm, a FR arm, and an arm housing. The arm driving unit 150 may be a component rotating and linearly moving the C-arc 140.

In addition, the arm driving unit 150 may be coupled to one side of the body 130. The arm driving unit 150 may be coupled to an upper side of the body 130. The arm driving unit 150 may be movable upward or downward with respect to the body 130. When the arm driving unit 150 moves upward or downward with respect to the body 130, the C-arc 140 may also move upward or downward with respect to the body 130 or the ground. In the present disclosure, a forward direction may be a direction from the body 130 toward the C-arc 140.

The arm driving unit 150 may include at least one of a stay arm 151, a front-rear driving arm 152, or an arm housing 153.

The stay arm 151 may be included in the arm driving unit 150. The stay arm 151 may be coupled to the C-arc 140 and may rotate the C-arc orbitally or pivotally. In FIG. 1, orbital rotation may mean that the C-arc 140 rotates about a rotation axis parallel to the rightward direction. Alternatively, orbital rotation may mean rotating the C-arc 140 in the longitudinal direction of the stay arm 151. In addition, orbital rotation may mean that the C-arc 140 rotates about a rotation axis perpendicular to the plane formed by the C-arc 140. Pivot rotation may mean that the C-arc 140 rotates about a rotation axis parallel to the forward direction. Pivot rotation is also referred to as rotational rotation. Pivot rotation may also be performed in the front-rear driving arm 152 rather than the stay arm 151.

The front-rear driving arm 152 may be included in the arm driving unit 150. The stay arm 151 may be coupled to one side of the front-rear driving arm 152. In FIG. 1, the front-rear driving arm 152 may be coupled to a rear side of the stay arm 151. The front-rear driving arm 152 may move the C-arc 140 in at least one of a forward direction, a rearward direction, an upward direction, and a downward direction. The front-rear driving arm 152 may also be a component for pivot-rotating the C-arc 140.

Referring to FIG. 1, the front-rear driving arm 152 may be coupled to the body 130 through a vertical movement column 154. The front-rear driving arm 152 may be movable upward or downward with respect to the vertical movement column 154. In addition, the front-rear driving arm 152 may be movable forward or rearward with respect to the vertical movement column 154. By the movement of the front-rear driving arm 152 with respect to the vertical movement column 154 or the body 130, the C-arc 140 may also be movable in at least one of upward, downward, forward, and rearward directions with respect to the body 130.

The arm housing 153 may be included in the arm driving unit 150. The arm housing 153 may be located above the front-rear driving arm 152. The arm housing 153 may be a component for coupling to at least one of the first display arm 160 and the second display arm 170. Components for coupling at least one of the first display arm 160 and the second display arm 170 to the body 130 may be located inside the arm housing 153. The arm housing 153 may be a component for protecting the components for coupling. The arm housing 153 may prevent foreign materials from being introduced into the components for coupling, thereby always allowing at least one of the first display arm 160 and the second display arm 170 to rotate smoothly.

Meanwhile, the radiation irradiator 110 may include a collimator and an X-ray tube.

The radiation irradiator 110 according to one embodiment may include an X-ray tube that receives a high voltage generated by the high-voltage generator and generates X-rays. The X-ray tube may be implemented as a two-pole vacuum tube having an anode and a cathode.

In addition, the radiation irradiator 110 may include a collimator that guides the path of X-rays irradiated from the X-ray tube and adjusts the irradiation area of the X-rays. The irradiation range of radiation may be determined by the collimator. The collimator may rotate with respect to the X-ray tube about an axis parallel to the irradiation direction of the radiation.

The high-voltage generator according to one embodiment may generate a high voltage for generating X-rays and apply the high voltage to the X-ray tube included in the radiation irradiator. The high-voltage generator may be included in the body 130, but the present disclosure is not limited thereto, and the high-voltage generator may be included in the radiation irradiator 110.

The radiation detector 120 according to one embodiment detects X-rays irradiated from the radiation irradiator and transmitted through the subject. The radiation detector may be a digital radiation detector. The radiation detector may be implemented using at least one of a thin film transistor (TFT), a charge coupled device (CCD), a complementary metal-oxide semiconductor (CMOS), computed radiography (CR), and film. The radiation detector may be included in the medical imaging apparatus 100 or may be a separate device that can be coupled to and detached from the medical imaging apparatus 100.

The medical imaging apparatus 100 may include a controller. In the present disclosure, the controller may mean at least one of a main controller included in the body 130, an irradiator controller included in the radiation irradiator, and a radiation detector controller included in the radiation detector. In addition, the controller may mean a controller included in a workstation external or internal to the medical imaging apparatus 100. The controller may be included in the first display 181 and the second display 182 rather than the body 130.

At least one of the first display 181 or the second display 182 may be a computing device controlling the operation of the medical imaging apparatus 100. That is, at least one of the first display 181 or the second display 182 may include a controller. At least one of the first display or the second display may be a touch display device controlling the operation of the medical imaging apparatus in a touch manner. That is, at least one of the first display or the second display may be included in the input unit.

At least one of the first display 181 or the second display 182 may be a display device outputting a captured radiation image. In addition, at least one of the first display 181 or the second display 182 may be a touch-input-capable operator console. In particular, it is preferable to use the first display 181 as an operator console. Using at least one of the first display 181 or the second display 182 that is an operator console, a user may set radiation conditions or imaging conditions. In addition, the user may recall previously stored radiation conditions or imaging conditions. The user may use at least one of the first display 181 or the second display 182 to set the positions of the radiation irradiator 110 and the radiation detector 120, imaging timing, imaging conditions, and the like.

The controller may control the operation of the medical imaging apparatus 100. For example, the controller may control the operation of at least one of the first display arm and the second display arm. In addition, the controller may control the radiation irradiator 110 to emit X-rays and control the radiation detector 120 to receive X-rays transmitted through the subject, thereby acquiring an X-ray image. According to one embodiment of the present disclosure, the controller may generate a medical image. For example, the controller may scan the radiation detector irradiated with X-rays to generate a medical image.

The controller may include one processor or may include a plurality of processors. The controller may be included in the body 130. When the controller includes a plurality of processors, at least some of the plurality of processors may be provided at a position physically spaced apart from the body 130. In addition, the medical imaging apparatus 100 is not limited thereto and may be implemented in various ways.

In addition, the medical imaging apparatus 100 may further include an operation unit providing an interface for operating the medical imaging apparatus 100. The operation unit may include an output unit and an input unit.

The output unit may output sound and images indicating imaging-related information such as X-ray irradiation or checking the status of the body under the control of the controller. The output unit may include a speaker or a display. The output unit may include at least one of the first display 181 and the second display 182. The output unit may output a medical image generated by the controller. The output unit may output information necessary for a user to operate the medical imaging apparatus 100, such as a user interface (UI), user information, or subject information. Examples of the output unit may include a speaker, a printer, a CRT display, an LCD display, a PDP display, an OLED display, a FED display, an LED display, a VFD display, a DLP display, an FPD display, a 3D display, a transparent display, and the like, and may include various output devices within a range apparent to those skilled in the art.

The input unit may receive commands for operating the medical imaging apparatus 100 and various information related to X-ray imaging from a user. The controller may control or operate the medical imaging apparatus 100 based on information input to the input unit. The input unit may include a joystick, a keyboard, a mouse, a touch screen, an imaging button, a locking release button, a voice recognizer, a fingerprint recognizer, an iris recognizer, a human motion recognizer, and the like, and may include input devices apparent to those skilled in the art. The input unit may include at least one of the first display 181 and the second display 182.

A user may input a command for X-ray irradiation through the input unit, and the input unit may be provided with a switch for inputting such a command. The switch may be provided such that an irradiation command for X-ray irradiation is input by pressing the switch at least once. For example, when the user presses the switch, a preparation command instructing preheating for X-ray irradiation may be input to the switch, and when the switch is pressed more deeply in that state, an irradiation command for actual X-ray irradiation may be input. When the user operates the switch in this way, the controller generates a signal corresponding to the command input through the switch operation, that is, a preparation signal, and transmits the preparation signal to the high-voltage generator that generates a high voltage for generating X-rays.

The high-voltage generator receives the preparation signal transmitted from the controller, starts preheating, and when preheating is completed, transmits a preparation completion signal to the controller. In addition, the radiation detector also needs to prepare for X-ray detection, and the controller transmits a preparation signal to the radiation detector so that the radiation detector can prepare to detect X-rays transmitted through the subject along with the preheating of the high-voltage generator. When the radiation detector receives the preparation signal, the radiation detector prepares to detect X-rays, and when the detection preparation is completed, the radiation detector transmits a detection preparation completion signal to the controller.

When the preheating of the high-voltage generator is completed and the X-ray detection preparation of the radiation detector is completed, the controller transmits an irradiation signal to the high-voltage generator, the high-voltage generator generates a high voltage and applies the high voltage to the X-ray tube, and the X-ray tube irradiates X-rays.

When transmitting the irradiation signal, the controller may transmit a sound or light output signal to the output unit so that the subject can be aware of the X-ray irradiation, so that a predetermined sound or light is output from the output unit. In addition, the output unit may output sound or light indicating other imaging-related information besides X-ray irradiation.

The controller controls the position of the X-ray irradiator and the radiation detector, imaging timing, and imaging conditions according to imaging conditions set by the user.

Specifically, the controller controls the irradiation timing of X-rays, the intensity of X-rays, and the irradiation area of X-rays by controlling the high-voltage generator and the radiation detector according to commands input through the input unit. In addition, the controller may adjust the position of the radiation detector according to predetermined imaging conditions and control the operation timing of the radiation detector.

In addition, the controller generates a medical image of the subject using image data received through the radiation detector. Specifically, the controller may receive image data from the radiation detector, remove noise from the image data, and adjust dynamic range and interleaving to generate a medical image of the subject.

The medical imaging apparatus 100 may include the first display 181 and the second display 182. The first display 181 may be coupled to the arm driving unit 150. The second display 182 may also be coupled to the arm driving unit 150. The first display 181 and the second display 182 may rotate independently of each other about the single arm rotation axis 190. The structure in which the first display 181 and the second display 182 are coupled to the arm driving unit 150 will be described below.

The medical imaging apparatus 100 may include the first display arm 160 and the second display arm 170. The first display arm 160 may be a component for coupling the first display 181 to the arm driving unit 150. In addition, the second display arm 170 may be a component for coupling the second display 182 to the arm driving unit 150.

The first display arm 160 and the second display arm 170 may rotate about the single arm rotation axis 190 so that the first display 181 and the second display 182 each rotate independently.

At least one of the first display arm 160 or the second display arm 170 may include at least one sub-arm. The second display arm 170 may include at least one sub-arm. For example, the second display arm 170 may include a base sub-arm 171 and a rotation sub-arm 172. The rotation sub-arm 172 may be rotatable with respect to the base sub-arm 171. The second display arm 170 may have a high degree of freedom of movement by the base sub-arm 171 and the rotation sub-arm 172. A user may place the second display 182 at a desired location while performing surgery or a procedure in a narrow space.

The medical imaging apparatus of the present disclosure may have a high degree of freedom of movement of a display arm coupled to a larger display. For example, one of the first display arm 160 and the second display arm 170 coupled to the larger display among the first display 181 and the second display 182 may have a high degree of freedom of movement. A high degree of freedom of movement may mean that the number of sub-arms is greater. Referring to FIG. 1, since the second display 182 is larger than the first display 181, the second display arm 170 coupled to the second display 182 may include two sub-arms, and the first display arm 160 may include one sub-arm. However, the present disclosure is not limited thereto, and the number of sub-arms may be independent of the size of the display.

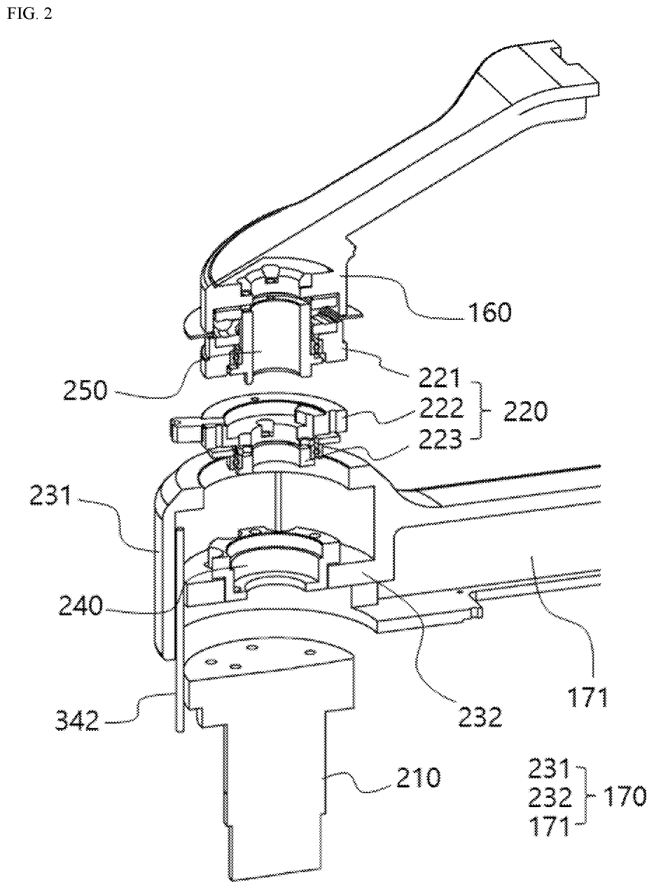

From FIG. 2 onward, the coupling of at least one of the first display arm 160 or the second display arm 170 to the arm driving unit 150 will be described.

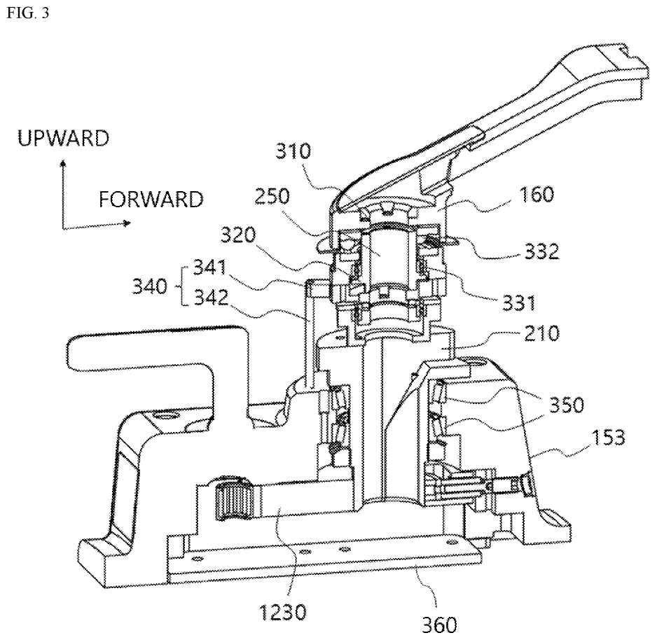

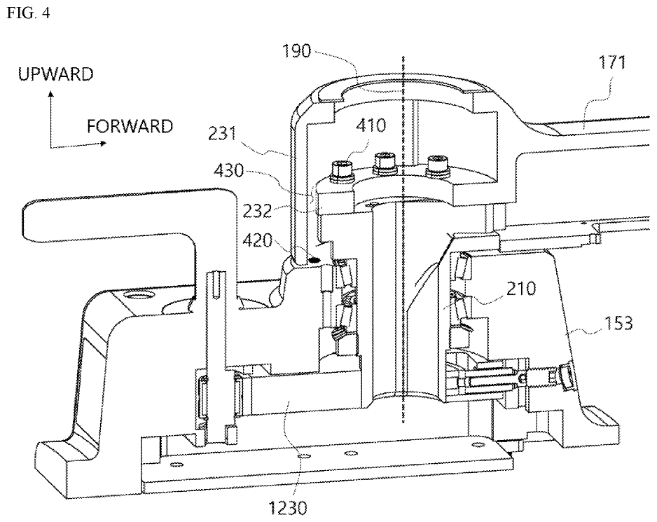

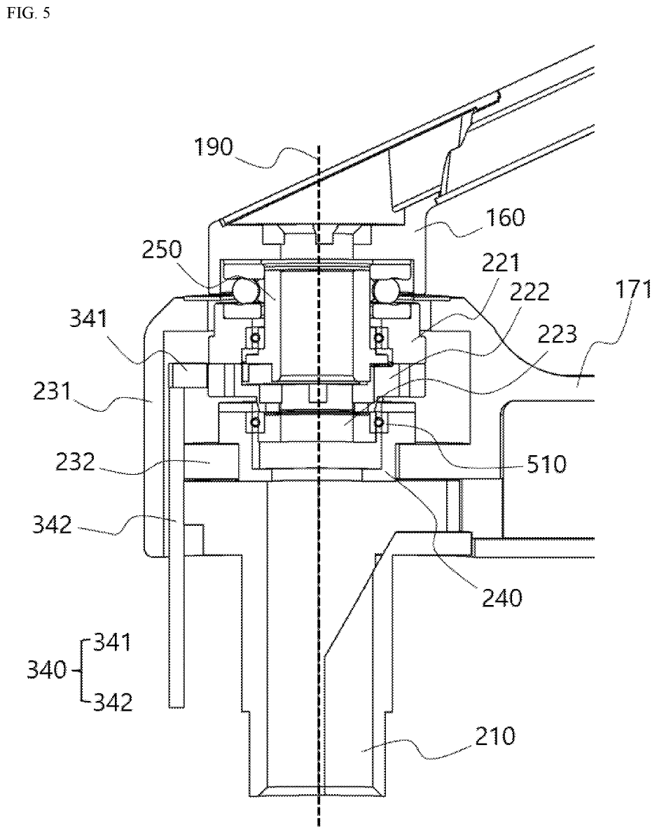

FIG. 2 illustrates a cross section of a part of the medical imaging apparatus according to one embodiment of the present disclosure. FIG. 3 illustrates a cross section of a part of the medical imaging apparatus according to one embodiment of the present disclosure. FIG. 4 illustrates a cross section of a part of the medical imaging apparatus according to one embodiment of the present disclosure. FIG. 5 illustrates a cross section of a part of the medical imaging apparatus according to one embodiment of the present disclosure.

Referring to FIGS. 2 to 5, the medical imaging apparatus 100 may include a display arm rotation pole 210. The display arm rotation pole 210 may be rotatably coupled to the arm driving unit 150. As described above, the arm driving unit 150 may include the arm housing 153. The display arm rotation pole 210 may be coupled to the arm housing 153 so as to be rotatable with respect to the arm housing 153. A fourth wear reduction member 350 may be located between the arm housing 153 and the display arm rotation pole 210.

At least a portion of the second display arm 170 may be coupled to the display arm rotation pole 210. The second display arm 170 may be fixed to the display arm rotation pole 210. More specifically, the second display arm 170 may be coupled to the display arm rotation pole 210 through a screw 410. However, the present disclosure is not limited thereto, and coupling methods other than screw coupling may also be used. In addition, the second display arm 170 may be formed integrally with the display arm rotation pole 210. The second display arm 170 may be rotatable with respect to the arm housing 153 together with the display arm rotation pole 210.

At least a portion of the first display arm 160 may be rotatably coupled to the second display arm 170. In this regard, the first display arm 160 and the second display arm 170 are not directly coupled to each other but may be coupled through a support block 220.

At least one of the first display arm 160 and the second display arm 170 may be rotatably coupled to the support block 220. The support block 220 may not be rotatable. At least one of the first display arm 160 and the second display arm 170 may be rotatable with respect to the fixed support block 220. The support block fixing unit 340 may fix the support block 220 so that the support block 220 is not rotatable.

The first display arm 160 and the support block 220 may be rotatably coupled. The first display arm 160 may be located above the support block 220. The support block 220 may support the first display arm 160. A bearing may be located between the support block 220 and the first display arm 160. In addition, the support block 220 may be rotatably coupled to the second display arm 170. That is, a bearing may be located between the support block 220 and the second display arm 170.

More specifically, the medical imaging apparatus 100 may further include at least one of a first wear reduction member 332, a second wear reduction member 331, a third wear reduction member 510, and a fourth wear reduction member 350.

At least one of the first wear reduction member 332, the second wear reduction member 331, the third wear reduction member 510, and the fourth wear reduction member 350 may be a component for assisting stable rotation of an object about an axis. At least one of the first wear reduction member 332, the second wear reduction member 331, the third wear reduction member 510, and the fourth wear reduction member 350 may prevent energy loss and reduce the risk of damage. In addition, at least one of the first wear reduction member 332, the second wear reduction member 331, the third wear reduction member 510, and the fourth wear reduction member 350 may reduce wear and reduce vibration or noise during rotation.

The second wear reduction member 331 may be located between the support block 220 and an arm shaft 250 coupled to the first display arm 160. In addition, the third wear reduction member 510 may be located between the support block receiving part 240 and the support block 220. The fourth wear reduction member 350 may be located between the arm housing 153 and the display arm rotation pole 210. In addition, the first wear reduction member 332 may be located between at least a portion of a lower surface of the first display arm 160 and at least a portion of an upper surface of the support block 220. The first wear reduction member 332 may include at least one of a first bearing or a first bushing. The second wear reduction member 331 may include at least one of a second bearing or a second bushing. The third wear reduction member 510 may include at least one of a third bearing or a third bushing. The fourth wear reduction member 350 may include at least one of a fourth bearing or a fourth bushing.

The second display arm 170 may include at least one of a display arm support block cover 231, a display arm inner plate 232, and sub-arms 171 and 172.

A support block receiving part 240 for coupling the support block 220 may be formed in the display arm inner plate 232. The support block receiving part 240 may be coupled to the display arm inner plate 232. That is, the support block receiving part 240 may be coupled to the second display arm 170.

The support block receiving part 240 may be coupled to the display arm inner plate 232 through a screw or adhesive or the like. However, the present disclosure is not limited thereto, and the display arm inner plate 232 may be formed integrally with the support block receiving part 240. The support block receiving part may be located at a lower portion of the second display arm 170. However, the present disclosure is not limited thereto, and the support block receiving part 240 may be located inside the second display arm 170.

The support block 220 may be rotatably coupled to the support block receiving part 240. The third support block 223 included in the support block 220 may be rotatably coupled to the support block receiving part 240. The third wear reduction member 510 may be located between the support block receiving part 240 and the support block 220.

Hereinafter, the support block 220 will be described in more detail.

The medical imaging apparatus 100 may include the support block 220. The support block 220 may be a component for independently rotating the first display arm 160 and the second display arm 170. The support block 220 may be fixed with respect to the arm housing 153. The first display arm 160 and the second display arm 170 may be independently rotatable with respect to the support block 220. That is, the second display arm 170 may rotate regardless of the rotation of the first display arm 160. In addition, the first display arm 160 may rotate regardless of the rotation of the second display arm 170.

The support block 220 may include one or more components. That is, the support block 220 may include at least one of a first support block 221, a second support block 222, and a third support block 223.

The first support block 221 may be a component for supporting at least a portion of a lower portion of the first display arm 160. The first display arm 160 may be rotatable with respect to the first support block 221. The first support block 221 may be rotatably coupled to the first display arm 160. A bearing may be located between the first support block 221 and the first display arm 160.

More specifically, a bearing may be located between the first support block 221 and the arm shaft 250 coupled to the first display arm 160. A screw 310 may pass through an upper side of the first display arm 160 and be coupled to the arm shaft 250 located at a lower side of the first display arm 160. The first display arm 160 and the arm shaft 250 may be formed integrally. The first display arm 160 and the arm shaft 250 are fixed, and the arm shaft 250 may rotate about the arm rotation axis 190 together with the first display arm 160.

At least a portion of the arm shaft 250 may have a cylindrical shape. At least a portion of an outer peripheral surface of the arm shaft 250 and at least a portion of an inner peripheral surface of a hole formed in the first support block 221 may face each other. The second wear reduction member 331 may be located between the outer peripheral surface of the arm shaft 250 and the inner peripheral surface of the hole formed in the first support block 221. The second wear reduction member 331 may be located between the first support block 221 and the arm shaft 250 coupled to the first display arm 160. The second wear reduction member 331 may include at least one of a second bearing or a bushing. When the second wear reduction member 331 is a second bearing, an outer ring of the second wear reduction member 331 may be in contact with an inner peripheral surface of the first support block 221, and an inner ring of the second wear reduction member 331 may be in contact with the outer peripheral surface of the arm shaft 250.

The first support block 221 may have a shape surrounding at least a portion of the outer peripheral surface of the arm shaft 250. At least a portion of the arm shaft 250 may be located above the second support block 222. The arm shaft 250 may be located above the third support block 223. The arm shaft 250 may be located above the display arm rotation pole 210. The outer peripheral surface of the arm shaft 250 may not face an inner peripheral surface or an outer peripheral surface of the first display arm rotation pole 730. The arm shaft 250 may be independently rotatable with respect to the display arm rotation pole 210.

A portion of the outer peripheral surface of the arm shaft 250 may include an arm shaft extension 320 extending in a radial direction. Since the arm shaft 250 has a cylindrical shape extending vertically and includes the arm shaft extension 320, the first display arm 160 may not wobble. The first display arm 160 may have only movement about the arm rotation axis 190. Therefore, the first display arm 160 may stably support the first display 181.

In addition, the first wear reduction member 332 may be located between at least a portion of a lower surface of the first display arm 160 and at least a portion of an upper surface of the first support block 221. Since bearings 331 and 332 are located between the first display arm 160 and the first support block 221, the first display arm 160 may stably rotate about the arm rotation axis 190 without wobbling. In addition, the arm shaft 250 coupled to the first display arm 160 may be independently rotatable with respect to the first display arm rotation pole 730.

The second support block 222 may be coupled to a lower portion of the first support block 221. The second support block 222 may be formed integrally with the first support block 221. The second support block 222 may be screw-coupled to the first support block 221. However, the present disclosure is not limited thereto, and coupling methods other than screw coupling may also be used. The second support block 222 may be formed integrally with the first support block 221.

The medical imaging apparatus 100 may include a support block fixing unit 340 for fixing the second support block 222 so that the second support block 222 is not rotatable with respect to the arm housing 153. The support block fixing unit 340 may be fixed to the arm housing 153. The support block fixing unit 340 may be coupled to the second support block 222 to fix the second support block 222 with respect to the arm housing 153. Since the second support block 222 is fixed with respect to the arm housing 153, the first support block 221 and the third support block 223 may also be fixed with respect to the arm housing 153.

The support block fixing unit 340 may include a support block fixing shaft 342 and a support block fixing hook 341.

The support block fixing shaft 342 may be fixed to at least one of the body 130 and the arm driving unit 150. The support block fixing shaft 342 may be fixed to at least a portion of a non-rotating component included in at least one of the body 130 and the arm driving unit 150. Here, the non-rotating component refers to a component that is fixed with respect to at least one of the first display arm 160 and the second display arm 170. At least one of the first display arm 160 and the second display arm 170 may be rotatable with respect to at least a portion of a non-rotating component included in at least one of the body 130 and the arm driving unit 150. The non-rotating component included in at least one of the arm driving units may include at least one of the stay arm 151, the front-rear driving arm 152, or the arm housing 153. For example, the support block fixing shaft 342 may be fixed to a lower plate 360 which is a non-rotating component included in at least one of the body 130 and the arm driving unit 150.

According to one embodiment of the present disclosure, the support block fixing shaft 342 may be inserted into and fixed to a shaft fixing hole 420 formed in the arm housing 153. The support block fixing shaft 342 may not be rotatable. The support block fixing shaft 342 may extend to be fixed to the arm housing 153. The support block fixing shaft 342 may extend upward from the arm housing 153. The support block fixing shaft 342 may be located outside the display arm rotation pole 210.

The support block fixing hook 341 may be coupled to the support block fixing shaft 342 and the second support block 222. The support block fixing hook 341 may be a component for preventing rotation of the second support block. For example, the support block fixing shaft 342 may be coupled to a shaft coupling part formed in the support block fixing hook 341. The support block fixing hook 341 may not move due to the support block fixing shaft 342. The support block fixing hook 341 may be coupled to the second support block 222. For example, a portion of the support block fixing hook 341 may be inserted into the second support block 222. The second support block 222 may not rotate due to the support block fixing hook 341. That is, even when the first display arm 160 and the second display arm 170 rotate, the second support block 222 may not rotate. The first display arm 160 and the second display arm 170 are independently rotatable.

Since the second support block 222 is coupled to the first support block 221 and the third support block 223, the fact that the second support block 222 is fixed with respect to the arm housing 153 may mean that the support block 220 is fixed with respect to the arm housing 153. Even when the first display arm 160 and the second display arm 170 rotate, the support block 220 may not rotate.

The support block fixing unit 340 is not limited to the description above. The support block fixing unit 340 may or may not be in contact with the support block 220 based on a user's input. The controller may control the support block fixing hook 341 of the support block fixing unit 340 to be in contact with or not in contact with the support block 220 based on a user's input or a predetermined control signal. When the support block fixing unit 340 is in contact with the support block 220, the support block 220 may not be rotatable. However, when the support block fixing unit 340 is not in contact with the support block 220, the support block 220 may be rotatable.

More specifically, when the user's input indicates independent rotation of the first display arm 160 and the second display arm 170, the controller may cause the support block fixing unit 340 to be in contact with the support block 220. In addition, when the user's input indicates dependent rotation of the first display arm 160 and the second display arm 170, the controller may control the support block fixing unit 340 not to be in contact with the support block 220. By controlling the support block fixing unit 340 in this way, the medical imaging apparatus 100 may enable various operations of the display arms. Therefore, user convenience may be increased.

The third support block 223 may be located at a lower portion of the second support block 222. The third support block 223 may be coupled to the lower portion of the second support block 222. The third support block 223 may be formed integrally with the second support block 222. The third support block 223 may be screw-coupled to the second support block 222. However, the present disclosure is not limited thereto, and coupling methods other than screw coupling may also be used.

The second display arm 170 may be rotatably coupled to the third support block 223. More specifically, the third wear reduction member 510 may be located between the second display arm 170 and the third support block 223. The third wear reduction member 510 may include at least one bearing. When the third wear reduction member 510 includes a plurality of bearings, rotation centers of the plurality of third wear reduction members may be located on the arm rotation axis 190. One bearing among the plurality of third wear reduction members may be located above, and the remaining bearings may be located below.

When the third wear reduction member 510 is a bearing, an inner ring of the third wear reduction member 510 may be disposed on an outer peripheral surface of the third support block 223, and an outer ring of the third wear reduction member 510 may be disposed on the second display arm 170. More specifically, the outer ring of the third wear reduction member 510 may be disposed in the support block receiving part 240 included in the second display arm 170. That is, the third wear reduction member 510 may be located between the support block receiving part 240 and the third support block 223. The support block receiving part 240 may include a bracket for fixing the outer ring of the third wear reduction member 510. Based on the bracket included in the support block receiving part 240, the outer ring of the third wear reduction member 510 may not be separated from the support block receiving part 240. A plurality of bearing balls may be located between the inner ring and the outer ring of the third wear reduction member 510.

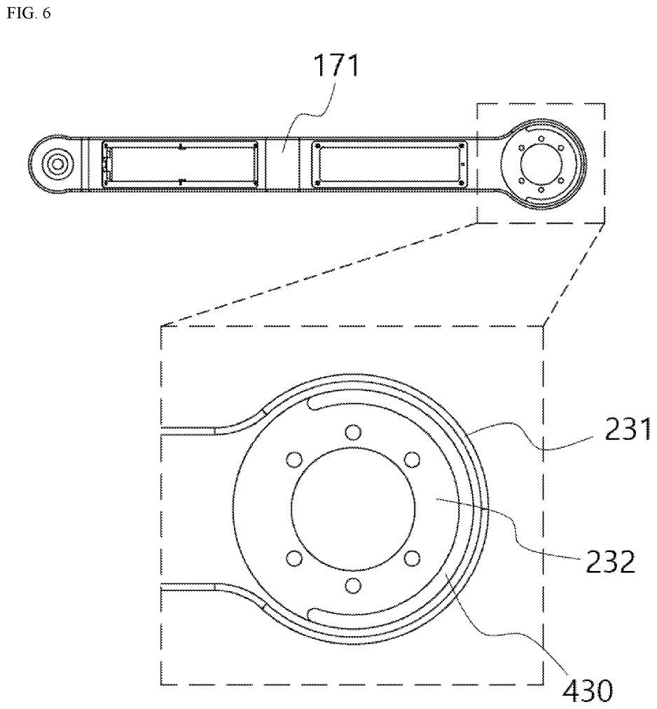

FIG. 6 is a view for describing the second display arm according to one embodiment of the present disclosure.

Referring to FIGS. 1, 4, and 6, the second display arm 170 may include at least one of a base sub-arm 171, a rotation sub-arm 172, a display arm support block cover 231, and a display arm inner plate 232. Since the base sub-arm 171 and the rotation sub-arm 172 have already been described above, the following description will focus on the display arm support block cover 231 and the display arm inner plate 232.

The display arm support block cover 231 may be a component for protecting at least one of the first support block 221, the second support block 222, and the third support block 223. That is, at least one of the first support block 221, the second support block 222, and the third support block 223 may be located inside the display arm support block cover 231. The first support block 221, the second support block 222, and the third support block 223 are components for determining the movement of the display arm, and may need to be protected from foreign materials. The display arm support block cover 231 included in the second display arm 170 protects the first support block 221, the second support block 222, the third support block 223, and components related to the movement of the display arm from foreign materials, thereby having the effect of increasing the durability of the display arm.

The display arm inner plate 232 may be located inside the display arm support block cover 231. The display arm inner plate 232 may be a component supporting at least a portion of a lower portion of the third support block 223. The display arm inner plate 232 may be coupled to at least a portion of the display arm support block cover 231. A portion of an outer peripheral surface of the display arm inner plate 232 may be coupled to a portion of an inner peripheral surface of the display arm support block cover 231. Referring to FIGS. 4 and 6, a display arm rotation limiting hole 430 extending at a predetermined limiting angle may be formed between the inner peripheral surface of the display arm support block cover 231 and the outer peripheral surface of the display arm inner plate 232. The display arm rotation limiting hole 430 may be formed at a location where the inner peripheral surface of the display arm support block cover 231 and the outer peripheral surface of the display arm inner plate 232 are not coupled.

The support block fixing shaft 342 may extend upward from the arm housing 153 and pass through the display arm rotation limiting hole 430. In addition, the support block fixing shaft 342 may extend to the support block fixing hook 341. The display arm rotation limiting hole 430 may be an angle through which the second display arm 170 is rotatable. Rotation of the second display arm 170 may be limited by interference between the support block fixing shaft 342 and the display arm inner plate 232.

More specifically, as described above, the support block fixing shaft 342 may be fixed with respect to the arm housing 153. The second display arm 170 may rotate with respect to the arm housing 153 about the arm rotation axis 190. At this time, the support block fixing shaft 342 may relatively move along the display arm rotation limiting hole 430. However, when the support block fixing shaft 342 meets the display arm inner plate 232, the second display arm 170 may no longer move. Therefore, the support block fixing shaft 342 and the display arm inner plate 232 may be components for limiting the rotation of the second display arm 170. The second display arm 170 may mount the relatively large second display 182. In addition, the second display arm 170 may be large, heavy, and long to support the second display 182. Since there may be a risk of an accident if the second display arm 170 moves to an unexpected position during surgery or a procedure, the movement of the second display arm 170 may be limited. In addition, the second display arm 170 may not come into contact with the first display 181 or the C-arc 140.

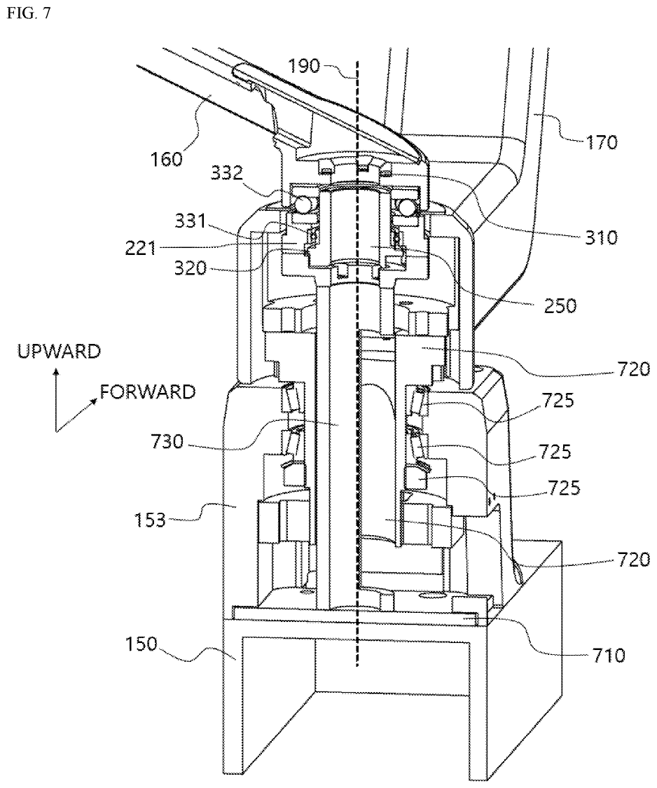

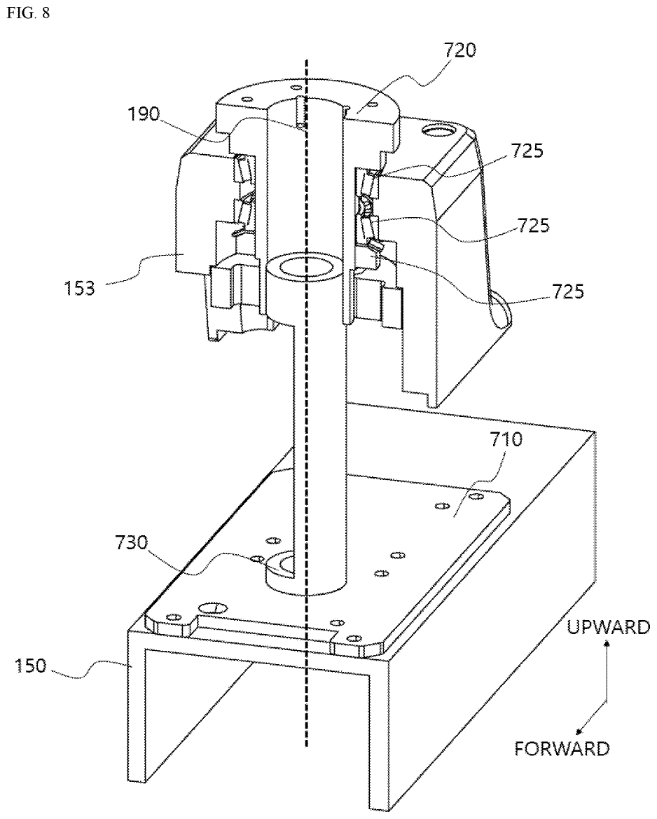

FIG. 7 is a view for describing the display arm according to one embodiment of the present disclosure. FIG. 8 is a view for describing the display arm according to one embodiment of the present disclosure.

The first display arm 160 and the second display arm 170 of FIG. 7 perform the same operations as those of FIGS. 2 to 6, but may include a structure different from that of FIGS. 2 to 6.

Referring to FIGS. 7 and 8, the medical imaging apparatus 100 may include a rotation pole fixing plate 710 coupled to an upper side of the arm driving unit 150. In addition, the medical imaging apparatus 100 may include an arm housing 153 coupled to at least one of the upper side of the arm driving unit 150 and the rotation pole fixing plate 710. At least a portion of the arm housing 153 may have a tubular shape. The inside of the arm housing 153 may be hollow. The arm driving unit 150 and the arm housing 153 may be separate components, or the arm housing 153 may be a component included in the arm driving unit 150. The arm housing 153 may be formed integrally with the arm driving unit 150.

When the first display arm rotation pole 730 is fixed directly to the arm driving unit 150 without the rotation pole fixing plate 710, there was a disadvantage that disassembly was difficult when a problem occurred with the arm driving unit 150. Therefore, it was difficult to identify the problem with the arm driving unit 150. Since the medical imaging apparatus 100 includes the rotation pole fixing plate 710 and the first display arm rotation pole 730 is coupled on top of the rotation pole fixing plate 710, there is an advantage that disassembly of the arm driving unit 150 becomes easier. Therefore, maintenance of the arm driving unit 150 may become easier. In particular, maintenance of the front-rear driving arm 152 is greatly facilitated.

The medical imaging apparatus 100 may include a second display arm rotation tube 720. At least a portion of the second display arm rotation tube 720 may have a tubular shape. The inside of the second display arm rotation tube 720 may be hollow. At least a portion of the second display arm rotation tube 720 may be inserted into a hole formed in the arm housing 153. At least a portion of the second display arm rotation tube 720 may be located inside the hole formed in the arm housing 153. At least a portion of the second display arm rotation tube 720 may be surrounded by the arm housing 153. The second display arm rotation tube 720 may be rotatably coupled to an inner peripheral surface of the arm housing 153. A bearing 725 may be located between the second display arm rotation tube 720 and the arm housing 153. A bearing 725 may be located between an outer peripheral surface of the second display arm rotation tube 720 and the inner peripheral surface of the arm housing 153.

The medical imaging apparatus 100 may include the first display arm rotation pole 730. A topmost end of the first display arm rotation pole 730 may be higher than a topmost end of the second display arm rotation tube 720. That is, the first display arm 160 may be rotatable at a higher position than the second display arm 170. Since the first display 181 coupled to the first display arm 160 is smaller than the second display 182, the heights of the first display 181 and the second display 182 can be approximately matched by allowing the first display arm 160 to rotate at a higher position. Therefore, the user can check the first display 181 and the second display 182 without having to move their eyes significantly.

At least a portion of the first display arm rotation pole 730 may be inserted into a hole formed in the second display arm rotation tube 720. An outer peripheral surface of the first display arm rotation pole 730 may face an inner peripheral surface of the second display arm rotation tube 720. At least a portion of the first display arm rotation pole 730 may be located inside the hole formed in the second display arm rotation tube 720. At least a portion of the first display arm rotation pole 730 may be surrounded by the second display arm rotation tube 720. An inner peripheral surface of the first display arm rotation pole 730 may not be in contact with the inner peripheral surface of the second display arm rotation tube 720. However, the present disclosure is not limited thereto. Rotation of the second display arm rotation tube 720 may be limited by a protrusion formed on the inner peripheral surface of the second display arm rotation tube 720 and a unique shape of the first display arm rotation pole 730. The first display arm rotation pole 730 may have a shape that is partially tubular, with a predetermined arc extending vertically. While the second display arm rotation tube 720 is rotating, when the first display arm rotation pole 730 comes into contact with the protrusion on the inner peripheral surface of the second display arm rotation tube 720, rotation of the second display arm rotation tube 720 may be prevented. According to the limitation of rotation of the second display arm rotation tube 720, rotation of the second display arm 170 coupled to the second display arm rotation tube 720 may also be limited.

One side of the first display arm rotation pole 730 may be fixed to the rotation pole fixing plate 710. That is, a lower side of the first display arm rotation pole 730 may be fixed to the rotation pole fixing plate 710. More specifically, the first display arm rotation pole 730 and the rotation pole fixing plate 710 may be coupled through a screw 410. However, the present disclosure is not limited thereto, and coupling methods other than screw coupling may also be used. In addition, the first display arm rotation pole 730 and the rotation pole fixing plate 710 may be formed integrally.

The first display arm rotation pole 730 extends upward, and the first display arm 160 may be rotatably coupled to the other side of the first display arm rotation pole 730. That is, the first display arm 160 may be coupled to an upper side of the first display arm rotation pole 730. The first display arm 160 may be rotatable at the upper side of the first display arm rotation pole 730.

More specifically, in FIG. 7, the first support block 221 may be fixed to the first display arm rotation pole 730. Specifically, the upper side of the first display arm rotation pole 730 may be coupled to a lower side of the first support block 221 through a screw 410. However, the present disclosure is not limited thereto, and coupling methods other than screw coupling may also be used. In addition, the first display arm rotation pole 730 and the first support block 221 may be formed integrally.

Bearings 331 and 332 may be located between the first display arm rotation pole 730 and the arm shaft 250 coupled to the first display arm 160. The first support block 221 may be coupled to the upper side of the first display arm rotation pole 730. Bearings 331 and 332 may be located between the first support block 221 and the arm shaft 250 coupled to the first display arm 160. A screw 310 may pass through an upper side of the first display arm 160 and be coupled to the arm shaft 250 located below the first display arm 160. The first display arm 160 and the arm shaft 250 may be formed integrally.

The first display arm 160 and the arm shaft 250 are fixed, and the arm shaft 250 may rotate about the arm rotation axis 190 together with the first display arm 160. At least a portion of the arm shaft 250 may have a cylindrical shape. At least a portion of an outer peripheral surface of the arm shaft 250 and at least a portion of an inner peripheral surface of a hole formed in the first support block 221 may face each other. The second wear reduction member 331 may be located between the first support block 221 and the arm shaft 250 coupled to the first display arm 160. That is, the second wear reduction member 331 may be located between the outer peripheral surface of the arm shaft 250 and the inner peripheral surface of the hole formed in the first support block 221. The second wear reduction member 331 may be located outside the arm shaft 250. The second wear reduction member 331 may be in contact with the arm shaft 250. However, the present disclosure is not limited thereto, and the second wear reduction member 331 may not be in contact with the arm shaft 250.

The arm shaft 250 may be located above the first display arm rotation pole 730. The outer peripheral surface of the arm shaft 250 may not face the inner peripheral surface or the outer peripheral surface of the first display arm rotation pole 730. The arm shaft 250 may be rotatable with respect to the fixed first display arm rotation pole 730.

A portion of the outer peripheral surface of the arm shaft 250 may include an arm shaft extension 320 extending in a radial direction. Since the arm shaft 250 has a cylindrical shape extending vertically and includes the arm shaft extension 320, the first display arm 160 may not wobble. The first display arm 160 may have only movement about the arm rotation axis 190. Therefore, the first display arm 160 may stably support the first display 181. In addition, the arm shaft 250 coupled to the first display arm 160 may be rotatable with respect to the fixed first display arm rotation pole 730.

In addition, the first wear reduction member 332 may be located between at least a portion of a lower surface of the first display arm 160 and at least a portion of an upper surface of the first support block 221. Since bearings 331 and 332 are located between the first display arm 160 and the first support block 221, the first display arm 160 may stably rotate about the arm rotation axis 190 without wobbling.

The second display arm rotation tube 720 may be rotatable about the arm rotation axis with respect to the arm housing 153. As described above, since the bearing 725 is located between the second display arm rotation tube 720 and the arm housing 153, the second display arm rotation tube 720 may rotate smoothly. Since the second display arm rotation tube 720 and the second display arm 170 are fixed, the second display arm 170 may also rotate with respect to the arm housing 153. The second display arm rotation tube 720 may be rotatable with respect to the fixed first display arm rotation pole 730.

In addition, the first display arm 160 may be rotatable about the arm rotation axis 190 with respect to the first display arm rotation pole 730. The first display arm rotation pole 730 is fixed to the rotation pole fixing plate 710, and the rotation pole fixing plate 710 may be fixed to the arm driving unit 150 including the arm housing 153. Therefore, the first display arm 160 may be rotatable with respect to the arm housing 153.

The first display arm 160 and the second display arm 170 may be independently rotatable. This is because the first display arm 160 is rotatably coupled to the first display arm rotation pole 730, and the second display arm 170 is coupled to the second display arm rotation tube 720 rotatably coupled to the arm housing 153.

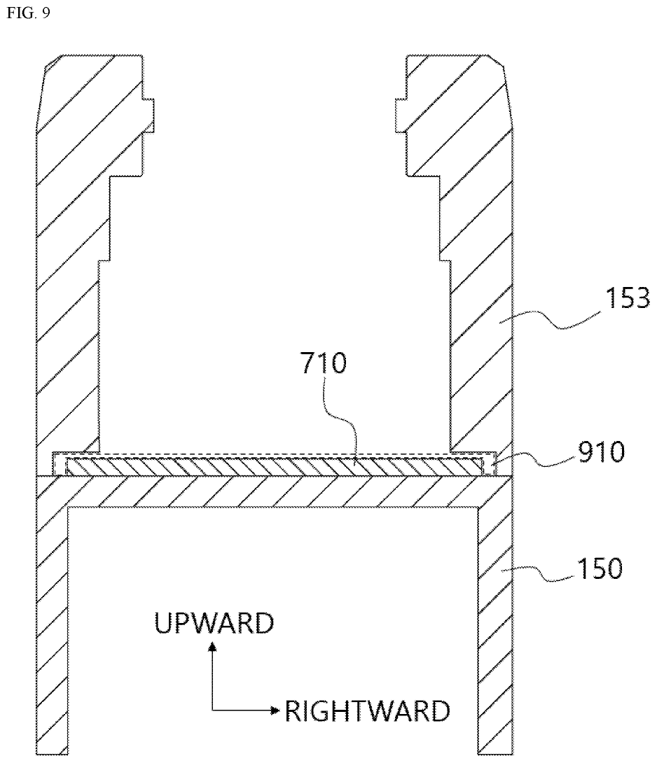

FIG. 9 is a view for describing the display arm according to one embodiment of the present disclosure.

The rotation pole fixing plate 710 may not be in contact with the arm housing 153. A space 910 may be formed between the rotation pole fixing plate 710 and the arm housing 153. The rotation pole fixing plate 710 and the arm housing 153 may not be directly coupled. The rotation pole fixing plate 710 and the arm housing 153 may be coupled through the arm driving unit 150.

In this way, since the medical imaging apparatus 100 of the present disclosure has the space 910 formed between the rotation pole fixing plate 710 and the arm housing 153, there is an effect of increasing the lifespan of the display arm. More specifically, due to the space 910 between the arm housing 153 and the rotation pole fixing plate 710, even when the arm housing 153 receives an impact from an external material, the impact may not be directly transmitted to the rotation pole fixing plate 710. In particular, when the arm housing 153 is made of an elastic material, all external impacts may be absorbed. Since the rotation pole fixing plate 710 is protected from external impacts, the first display arm rotation pole 730 coupled to the rotation pole fixing plate 710 may also be protected from impacts. That is, the first display arm 160 may stably rotate with respect to the firmly fixed first display arm rotation pole 730, and the lifespan may also be increased.

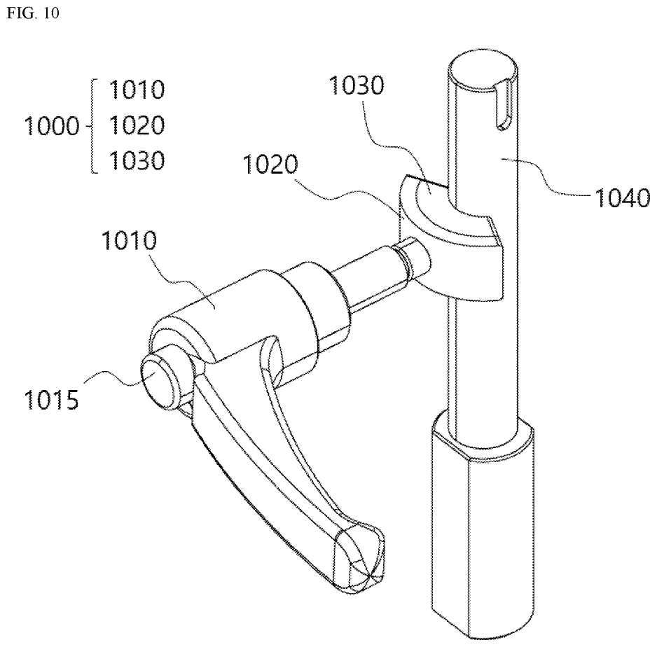

FIG. 10 is a view for describing the movement fixing unit according to one embodiment of the present disclosure.

The medical imaging apparatus 100 of the present disclosure may further include a movement fixing unit 1000. The movement fixing unit 1000 may be a component for limiting at least one of rotation of the C-arc, rotation of the first display arm, and rotation of the second display arm. Limiting rotation by the movement fixing unit 1000 may mean preventing rotation, preventing sudden movement, or maintaining the posture of the medical imaging apparatus 100 when the user does not apply force.

Rotation of the C-arc 140 may include at least one of orbital rotation or pivot rotation of the C-arc. In addition, movement of the C-arc in at least one of upward-downward movement, forward-rearward movement, leftward-rightward movement, or wig-wag movement may also be limited by the movement fixing unit 1000.

Rotation of the first display arm 160 may mean rotation of the first display arm 160 about the arm rotation axis 190. However, the present disclosure is not limited thereto, and it may also mean rotation between sub-arms included in the first display arm 160.

Rotation of the second display arm 170 may mean rotation of the second display arm 170 about the arm rotation axis 190. In addition, rotation of the second display arm 170 may mean rotation of the rotation sub-arm 172 with respect to the base sub-arm 171.

The movement fixing unit 1000 may limit or not limit rotation in a one-touch manner. The movement fixing unit 1000 may include a clamp lever device that limits or does not limit rotation of a rotation target 1040. When the clamp lever device is tightened, rotation of the rotation target 1040 may be limited, and when the clamp lever device is not tightened, rotation of the rotation target 1040 may not be limited. The movement fixing unit 1000 including the clamp lever device may include at least one of a lever 1010, a brake shoe 1020, and a brake lining 1030. The clamp lever device being tightened means that the lever 1010 rotates in one direction so that the brake lining 1030 contacts the rotation target 1040 at high pressure, and the clamp lever device not being tightened means that the lever 1010 rotates in the other direction so that the brake lining 1030 contacts the rotation target 1040 at low pressure or does not contact the rotation target 1040.

Conventionally, in order to fix the display arm, the display was mounted on a handle such as a parking handle. At this time, a user had to hold the parking handle to control the medical imaging apparatus, and when the display was mounted on the parking handle, it could be difficult to grip the parking handle, and it was not easy to smoothly mount the display on the parking handle while holding the parking handle. In addition, the display arm could be stiff to withstand the weight of the display, and the user could find it difficult to control the display arm. In addition, when the display was mounted on the parking handle, there was a problem that the display and the parking handle collided and scratches were generated. In addition, when parking the display, there was a problem that the display was damaged by the impact of contact between the display and the parking handle.

Since the medical imaging apparatus of the present disclosure limits the movement of the display arm in a one-touch manner, the above problems can be resolved. More specifically, the medical imaging apparatus 100 of the present disclosure may include a lever 1010, and the lever 1010 may include a lever switch 1015. When the lever switch 1015 is pressed, the movement fixing unit 1000 may limit at least one of rotation of the C-arc, rotation of the first display arm, and rotation of the second display arm. In addition, when the lever switch 1015 is pressed once more, the movement fixing unit 1000 may not limit at least one of rotation of the C-arc, rotation of the first display arm, and rotation of the second display arm.

In addition, when the lever 1010 is rotated in one direction, the movement fixing unit 1000 may limit at least one of rotation of the C-arc, rotation of the first display arm, and rotation of the second display arm. In addition, when the lever 1010 is rotated in the other direction, the movement fixing unit 1000 may not limit at least one of rotation of the C-arc, rotation of the first display arm, and rotation of the second display arm.

However, the present disclosure is not limited thereto, and the medical imaging apparatus 100 may include a touch sensor. The touch sensor may be used instead of the lever switch 1015. When the user touches the touch sensor, the touch sensor transmits a signal to the controller, the controller transmits a control signal to the movement fixing unit 1000, and the movement fixing unit 1000 may limit at least one of rotation of the C-arc, rotation of the first display arm, and rotation of the second display arm. When the user touches the touch sensor again, the touch sensor transmits a signal to the controller, the controller transmits a control signal to the movement fixing unit 1000, and the movement fixing unit may not limit at least one of rotation of the C-arc, rotation of the first display arm, and rotation of the second display arm.

The medical imaging apparatus may include at least one of a lever 1010, a lever switch 1015, or a touch sensor. The lever, lever switch 1015, or touch sensor is located near a component that rotates, so the user can intuitively know which movement the lever or touch sensor will limit.

In this way, the medical imaging apparatus 100 of the present disclosure can stop the movement of the medical imaging apparatus 100 with a single operation and can maintain the posture of the medical imaging apparatus 100. In addition, the user can quickly and easily fix the display by fixing the display arm.

Referring to FIG. 10, the movement fixing unit 1000 may include a brake lining 1030 and a brake shoe 1020.

The brake lining 1030 may be a component that contacts a rotation target 1040 and prevents rotation of the rotation target. The brake lining 1030 may be a woven liner woven from fiber material. The brake lining 1030 may be a woven liner made in a lining form by weaving copper wire or fiber material (including glass fiber, etc.) and then repeatedly impregnating with a lining solution, drying, and heat-treating. Unlike a molded lining, such a brake lining 1030 has appropriate flexibility and is not significantly affected by the R value of the rotation target, and thus has the advantage of being able to be disposed and used at various types of positions, rotation axes, and the like. In addition, when grease is applied to the contact surface between the rotation target and the brake lining 1030, noise and wear on the friction surface can be further reduced.

The rotation target 1040 may refer to a component that rotates relative to the movement fixing unit 1000. When a first component and a second component rotate relative to each other, the movement fixing unit 1000 may be included in the first component, and the rotation target 1040 may be included in the second component. When the second component is a rotation rotor serving as a rotation axis, the brake lining 1030 of the movement fixing unit 1000 included in the first component may contact the rotation rotor, and rotation of the first component and the second component may be limited. When the brake lining 1030 of the movement fixing unit 1000 does not contact the rotation rotor, rotation of the first component and the second component may not be limited.

The brake shoe 1020 may be coupled to at least a portion of the brake lining 1030. The brake shoe 1020 may be a component that moves the brake lining 1030 toward the rotation target 1040 or away from the rotation target. The brake shoe 1020 may be coupled to a push rod. When the lever switch 1015 is pressed by the user, the brake shoe 1020 may move toward the rotation target 1040 by the push rod. That is, rotation may be limited. In addition, when the lever switch 1015 is pressed once more, the brake shoe 1020 may move away from the rotation target 1040 by the push rod. When the lever 1010 rotates in one direction, the brake shoe 1020 may move toward the rotation target 1040 by the push rod. In addition, when the lever 1010 rotates in the other direction, the brake shoe 1020 may move away from the rotation target 1040 by the push rod.

The brake lining 1030 may be made of a soft material. Therefore, it may be difficult for the brake lining 1030 alone to closely contact the rotation target 1040. Therefore, the brake shoe 1020 may be a component for causing the brake lining 1030 to closely contact the rotation target 1040. The brake shoe 1020 may be made of a rigid material. The brake shoe 1020 may be formed to surround a portion or all of a periphery of the brake lining 1030. The brake shoe 1020 may press the brake lining 1030 so that the brake lining 1030 contacts the rotation target 1040 over a wide area.

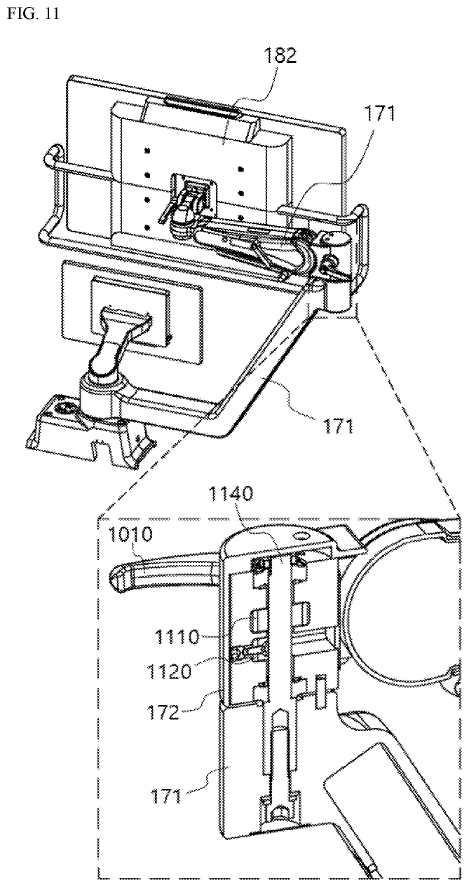

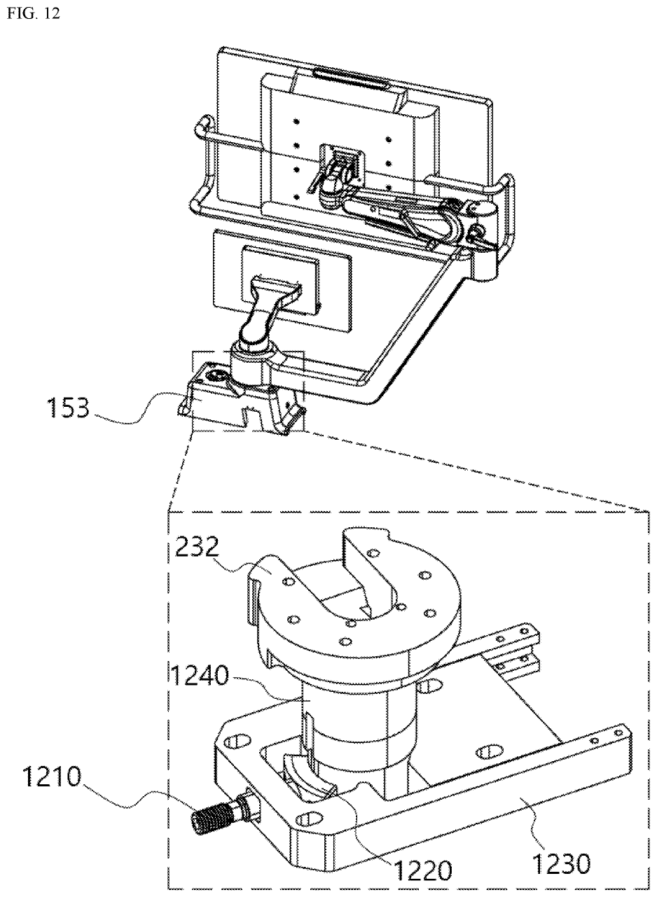

FIG. 11 is a view for describing the movement fixing unit according to one embodiment of the present disclosure. FIG. 12 is a view for describing the movement fixing unit according to one embodiment of the present disclosure.

FIGS. 11 and 12 show examples of positions where the movement fixing unit may be located. However, it should be understood that the movement fixing unit may be located at positions other than those shown in FIGS. 11 and 12.

Referring to FIG. 11, the base sub-arm 171 may be coupled to a rotation rotor 1140 which is the rotation target 1040. The rotation rotor 1140 may not rotate with respect to the base sub-arm 171. The rotation sub-arm 172 may rotate about the rotation rotor 1140 as an axis. The rotation sub-arm 172 may include a lever 1010. The brake lining 1110 included in the movement fixing unit 1000 may or may not contact the rotation rotor 1140 by pressing of the lever switch 1015 or rotation of the lever 1010. The brake lining 1110 may be included in the rotation sub-arm 172. Depending on whether the brake lining 1110 contacts the rotation rotor 1140, the rotation sub-arm 172 may or may not rotate with respect to the base sub-arm 171.

Referring to FIG. 11, the rotation sub-arm 172 may include a preload brake lining 1120. The preload brake lining 1120 may be in contact with the rotation rotor 1140. The pressure applied by the preload brake lining 1120 to the rotation rotor 1140 may be controlled by a preload adjustment screw. The greater the pressure applied by the preload brake lining 1120 to the rotation rotor 1140, the stiffer the rotation of the rotation sub-arm 172 with respect to the base sub-arm 171 may be. In addition, the smaller the pressure applied by the preload brake lining 1120 to the rotation rotor 1140, the smoother the rotation of the rotation sub-arm 172 with respect to the base sub-arm 171 may be. Based on the preload brake lining 1120, the medical imaging apparatus 100 has the effect of being able to maintain the posture desired by the user, the effect of being able to withstand the weight of the display, and the effect of not moving suddenly. For example, the weight of the display may be heavy, and the rotation sub-arm 172 may suddenly move with respect to the base sub-arm 171 due to the weight of the display. However, such sudden movement may be prevented by the preload brake lining 1120. In addition, the preload brake lining 1120 may smoothly surround the rotation rotor 1140 to reduce noise and wear.

Referring to FIG. 12, the arm driving unit 150 may include a vertical movement shaft 1240 for moving the C-arc 140 vertically. In FIG. 12, the vertical movement shaft 1240 may be the rotation target 1040. The arm driving unit 150 may include a brake plate 1230. A preload adjustment screw 1210 may be coupled to the brake plate 1230. The pressure applied by a preload brake lining 1220 to the vertical movement shaft 1240 may be controlled by the preload adjustment screw 1210. The greater the pressure applied by the preload brake lining 1220 to the vertical movement shaft 1240, the slower the vertical movement shaft 1240 may move upward or downward. In addition, the smaller the pressure applied by the preload brake lining 1220 to the vertical movement shaft 1240, the faster the vertical movement shaft 1240 may move upward or downward. Based on the preload brake lining 1220, the medical imaging apparatus 100 has the effect of being able to maintain the posture desired by the user, the effect of being able to withstand the weight of the C-arc 140, and the effect of not moving suddenly. For example, the weight of the C-arc 140 may be heavy, and the vertical movement shaft 1240 may suddenly move due to the weight of the C-arc 140. However, such sudden movement may be prevented by the preload brake lining 1220. The preload brake lining 1220 may smoothly surround the vertical movement shaft 1240 to reduce noise and wear.

Various embodiments have been described above. Those skilled in the art will understand that the present disclosure may be embodied in various other forms without departing from the essential characteristics of the present disclosure. Therefore, the disclosed embodiments should be considered in an illustrative rather than a restrictive sense. The scope of the present disclosure is defined by the appended claims rather than by the foregoing description, and all differences within the scope of equivalents thereof should be construed as being included in the present disclosure.

Meanwhile, the embodiments of the present disclosure described above may be written as a program executable on a computer and may be implemented in a general-purpose digital computer that operates the program using a computer-readable recording medium. The computer-readable recording medium includes storage media such as magnetic storage media (e.g., ROM, floppy disk, hard disk, etc.) and optically readable media (e.g., CD-ROM, DVD, etc.).

What is claimed is:

1. A medical imaging apparatus comprising:
a body;
an arm driving unit supporting and rotating a C-arc, and coupled to one side of the body;
a first display coupled to the arm driving unit; and
a second display coupled to the arm driving unit,
wherein the first display and the second display rotate independently of each other about a single arm rotation axis.

2. The medical imaging apparatus of claim 1, further comprising:
a first display arm 160 for coupling the first display to the arm driving unit; and
a second display arm 170 for coupling the second display to the arm driving unit,
wherein the first display arm and the second display arm rotate about the single arm rotation axis so that the first display and the second display each rotate independently.

3. The medical imaging apparatus of claim 2, wherein the second display arm 170 comprises at least one sub-arm.

4. The medical imaging apparatus of claim 2, comprising a display arm rotation pole coupled to an arm housing 153 included in the arm driving unit so as to be rotatable with respect to the arm housing,
wherein at least a portion of the second display arm is coupled to the display arm rotation pole, and
at least a portion of the first display arm is rotatably coupled to the second display arm.

5. The medical imaging apparatus of claim 4, further comprising:
a support block to which at least one of the first display arm and the second display arm is rotatably coupled; and
a support block fixing unit 340 for fixing the support block so that the support block is not rotatable.

6. The medical imaging apparatus of claim 5, further comprising:
a support block receiving part 240 coupled to the second display arm 170 and rotatably coupled to the support block;
a second wear reduction member 331 located between the support block and an arm shaft 250 coupled to the first display arm 160;
a third wear reduction member 510 located between the support block receiving part and the support block;
a fourth wear reduction member 350 located between the arm housing 153 and the display arm rotation pole 210; and
a first wear reduction member 332 located between at least a portion of a lower surface of the first display arm 160 and at least a portion of an upper surface of the support block 220.

7. The medical imaging apparatus of claim 5, wherein the support block comprises:
a first support block 221 for supporting at least a portion of a lower portion of the first display arm;
a second support block 222 coupled to a lower portion of the first support block; and
a third support block 223 located at a lower portion of the second support block and to which the second display arm is rotatably coupled.

8. The medical imaging apparatus of claim 2, further comprising:
a radiation irradiator outputting radiation and irradiating the radiation onto a subject;
a radiation detector receiving the radiation transmitted through the subject and acquiring an image signal;
a C-arc coupled to the radiation irradiator and coupled to the radiation detector at a position opposite to the radiation irradiator;
a stay arm included in the arm driving unit, coupled to the C-arc, and rotating the C-arc orbitally or pivotally;
a front-rear driving arm included in the arm driving unit, having the stay arm coupled to one side thereof, and moving the C-arc in at least one of a forward direction, a rearward direction, an upward direction, and a downward direction;
an arm housing included in the arm driving unit and located above the front-rear driving arm; and
a movement fixing unit for limiting at least one of rotation of the C-arc, rotation of the first display arm, and rotation of the second display arm.

9. The medical imaging apparatus of claim 8, wherein the movement fixing unit comprises:
a brake lining contacting a rotation target and preventing rotation of the rotation target; and
a brake shoe coupled to at least a portion of the brake lining and moving the brake lining toward the rotation target or away from the rotation target.

10. The medical imaging apparatus of claim 9, wherein the brake lining is a woven liner woven from fiber material.
